(19) United States
(12) Patent Application Publication
(10) Pub. No.: US 2026/0207964 A1
(43) Pub. Date: Jul. 23, 2026

(54) POSITION-VARIABLE LED LIGHT THERATY DEVICE

(71) Applicant: BENILIGHT CO., LTD., Cheonan-si, Chungcheongnam-do (KR)

(72) Inventor: Yo Sik SONG, Cheonan-si Chungcheongnam-do (KR)

(21) Appl. No.: 19/141,356

(22) PCT Filed: Jul. 27, 2023
(86) PCT No.: PCT/KR2023/010988

§ 371 Date: Oct. 28, 2025

(30) Foreign Application Priority Data
Dec. 20, 2022 (KR) 10-2022-0179367

(51) Int. Cl. A61N 5/06

(52) U.S. Cl. CPC A61N 5/0616; A61N 2005/0633; A61N 2005/0643; A61N 2005/0652; A61N 2005/0664

(57) ABSTRACT

A position-variable LED light therapy device includes an LED pad on which a plurality of LEDs are arranged, a pair of support units that supports the LED pad, a pair of base units in which the pair of support units are installed, and a fixing bar that secures the pair of base units spaced apart at a predetermined distance from each other, wherein the support unit is provided with a rotary unit in an upper portion thereof to adjust an installation angle of the LED pad, and the base unit is formed with a plurality of upper grooves on an upper portion thereof to insert and fix the support unit therein at various positions.

### TECHNICAL FIELD

The disclosure relates to a position-variable LED light therapy device, and more specifically to a position-variable LED light therapy device in which a plurality of LED pads are arranged and rotated to irradiate multiple beams of light onto a desired area of the human body.

### BACKGROUND ART

Light therapy is utilized for health management and treatment using the stimulating effects of light of specific wavelengths on the human body.

It is known that red light promotes blood circulation and alleviates congestion; vermilion light reduces inflammation and activates the functions of the brain and kidneys; orange light lowers insulin requirements; green light stimulates the sympathetic nervous system, thereby enhancing the functions of the kidneys or liver and having an detoxifying effect on harmful environmental substances; blue light is effective in stabilizing psychological states to alleviate insomnia, and also effective for asthma, respiratory diseases, muscular atrophy, digestive disorders, ulcers, and thyroid dysfunction; purple light is useful in the treatment of obesity due to its function of regulating appetite, and is also effective in stabilizing heart rate; pink light has a greater effect on mental aspects rather than physical ones and revitalizes both the mind and the body; yellow light has a therapeutic effect on the common cold; and deep red light is effective in relieving headaches and migraines and in promoting psychological stability.

A light emitting diode (LED) light therapy device is inconvenient to use for a long time while holding both ends of a pad with a plurality of LEDs attached thereto by hand, and thus employs a holder for securing the LED pad.

A conventional holder for the LED light therapy device has a securing-type design and allows only for angle adjustments, thereby making it difficult to irradiate light to multiple areas simultaneously or irradiate light of different colors.

### DOCUMENT OF RELATED ART

### Patent Document

(Patent Document 1) Korean Patent No. 10-1324255

### DISCLOSURE

### Technical Problem

The disclosure has been conceived to solve the foregoing problems of the related art, and an aspect of the disclosure is to provide a position-variable light emitting diode (LED) light therapy device that is convenient to irradiate multiple beams of light to various areas such as the face, head, neck, abdomen, and waist, and is capable of simultaneously irradiating light to multiple areas.

### Technical Solution

According to an embodiment of the disclosure, a position-variable light emitting diode (LED) light therapy device including an LED pad on which a plurality of LEDs are arranged, a pair of support units that supports the LED pad, a pair of base units in which the pair of support units are installed, and a fixing bar that secures the pair of base units spaced apart at a predetermined distance from each other, wherein the support unit is provided with a rotary unit in an upper portion thereof to adjust an installation angle of the LED pad, and the base unit is formed with a plurality of upper grooves on an upper portion thereof to insert and fix the support unit therein at various positions.

The rotary unit may include an upper protrusion formed to be inserted and fixed into a groove of the LED pad, and the support unit may include a lower protrusion formed at a lower end thereof to be press-fitted into the upper groove.

The LED pad may be configured to be separated from the rotary unit and to irradiate light manually to a desired area.

The LED pad may include a plurality of LED pads, and the plurality of LED pads may be different from each other in color of light irradiated from the LEDs arranged thereon.

The plurality of support units may be installed on the base unit to install the plurality of LED pads.

The rotary unit may include: a shaft installed in the support unit; a spin block rotatably installed on the shaft; a plurality of ball plungers installed in the spin block, and spaced apart from each other along the inner circumference of the spin block, each ball plunger including a casing, and a ball inserted and installed in the casing so as to be retractably by an elastic member; a ball spacer installed in the support unit to face the spin block and including a plurality of engaging grooves recessed along the circumference thereof to engage with the balls of the ball plungers, a thrust bearing put on the shaft and installed on the spin block so as to allow the spin block to spin, and a nut fastened to the shaft to prevent the thrust bearing from coming off.

The rotary unit may include a cover installed to cover the nut.

The shaft may be structured to include a threaded portion formed on one side of a shaft body, and a retaining protrusion formed at the other end.

The support unit may include a ring-shaped spacer groove formed in an upper portion of a support bar having a plate-like bar shape and recessed to receive the ball spacer, a shaft insertion hole formed at the center of the spacer groove to insert the shaft therein, and a retaining groove recessed in an outer portion of the shaft insertion hole of the support unit to receive the retaining protrusion.

The spin block may include a shaft insertion hole formed at the center of a block body, a plurality of plunger insertion portions formed along the inner circumference of the shaft insertion hole of the block body so as to insert the ball plungers therein, a bearing mounting groove recessed from the outer side of the block body to insert the thrust bearing therein, and the upper protrusion protrudes from the upper portion of the block body.

The base units are provided as a pair to face each other, and the base unit is formed with a pair of lower grooves in lower portions thereof to insert the fixing bar therein.

The lower groove is recessed in a T-shape.

### ADVANTAGEOUS EFFECTS

A position-variable light emitting diode (LED) light therapy device according to the disclosure is convenient to use for a certain period of time by irradiating multiple beams of light to various areas such as the face, head and neck, and provides therapeutic effects on multiple areas at once.

In addition, a position-variable LED light therapy device according to the disclosure employs a plurality of LED light therapy devices to irradiate light of different colors simultaneously, thereby providing a plurality of therapeutic effects.

Further, a position-variable LED light therapy device according to the disclosure provides therapeutic effects in such a manner that an LED pad is separated from a support unit and manually placed in position to irradiate light onto a desired area such as the chest, abdomen, waist, back, arm or leg.

### DESCRIPTION OF DRAWINGS

FIG. 1 is an exploded perspective view showing that a light emitting diode (LED) pad is separated in a position-variable LED light therapy device according to an embodiment of the disclosure.

FIG. 2 is a perspective view of a position-variable LED light therapy device according to an embodiment of the disclosure, excluding an LED pad.

FIG. 3 is a view illustrating that a rotation angle of an LED pad is adjusted in a position-variable LED light therapy device according to an embodiment of the disclosure.

FIG. 4 is a view showing a state of using a position-variable LED light therapy device according to an embodiment of the disclosure.

FIG. 5 is a side view showing another state of using a position-variable LED light therapy device according to an embodiment of the disclosure.

FIG. 6 is a perspective view showing another state of using a position-variable LED light therapy device according to an embodiment of the disclosure.

FIGS. 7A and 7B are enlarged exploded perspective views of a rotary unit in FIG. 2, in which FIG. 7A shows the rotary unit as seen from one side, and FIG. 7B shows the rotary unit as seen from the other side.

### BEST MODE

Below, descriptions will be made with reference to specific embodiments of the disclosure and the accompanying drawings. The embodiments of the disclosure are for illustrative purpose only, and the scope of the disclosure is not limited to these embodiments. Further, the accompanying drawings show only essential elements exaggeratedly for clarity without irrelevant elements, and thus the disclosure should not be construed as being limited to the accompanying drawings.

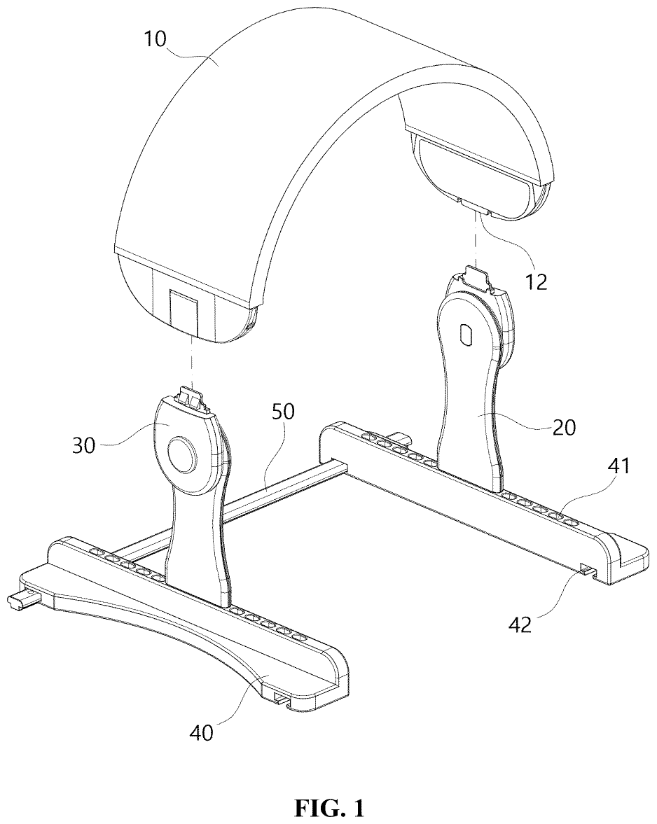

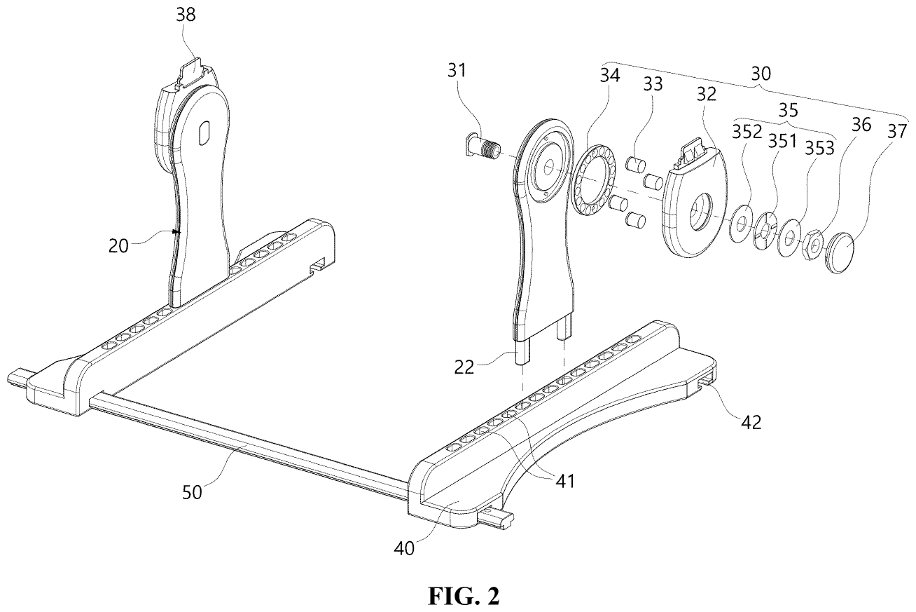

FIG. 1 is an exploded perspective view showing that an LED pad is separated in a position-variable (light emitting diode (LED) light therapy device according to an embodiment of the disclosure, and FIG. 2 is a perspective view of the position-variable LED light therapy device according to an embodiment of the disclosure, excluding the LED pad and showing an exploded perspective view of one of rotary units. In the following description, an inner side refers to a direction toward a support unit 20 and an outer side refers to a direction opposite to the support unit 20.

Referring to FIGS. 1 and 2, the position-variable LED light therapy device according to an embodiment of the disclosure includes an LED pad 10 on which a plurality of LEDs (not shown) are arranged, a pair of support units 20 that supports the LED pad 10, a pair of base units 40 in which the pair of support units 20 are installed, and a fixing bar 50 that secures the pair of base units 40 spaced apart at a predetermined distance from each other.

Further, the position-variable LED light therapy device according to an embodiment of the disclosure is characterized in that the support unit 20 is provided with a rotary unit 30 in an upper portion thereof to adjust an installation angle of the LED pad 10, and the base unit 40 is formed with a plurality of upper grooves 41 on an upper portion thereof to insert and fix the support unit 20 therein at various positions.

Referring to FIG. 2, the rotary unit 30 includes an upper protrusion 38 formed to be inserted and fixed into a groove 12 formed at each end of the LED pad 10, and the support unit 20 includes a pair of lower protrusions 22 at a lower end thereof to be press-fitted into the upper grooves 41 of the base unit 40.

The LED pad 10 is separated from the support units 20 by being pulled out from the upper protrusions 38 of the rotary units 30. The LED pad 10 separated in this way may be manually moved to irradiate light to a desired area such as the chest, abdomen, waist, back, arm, or leg.

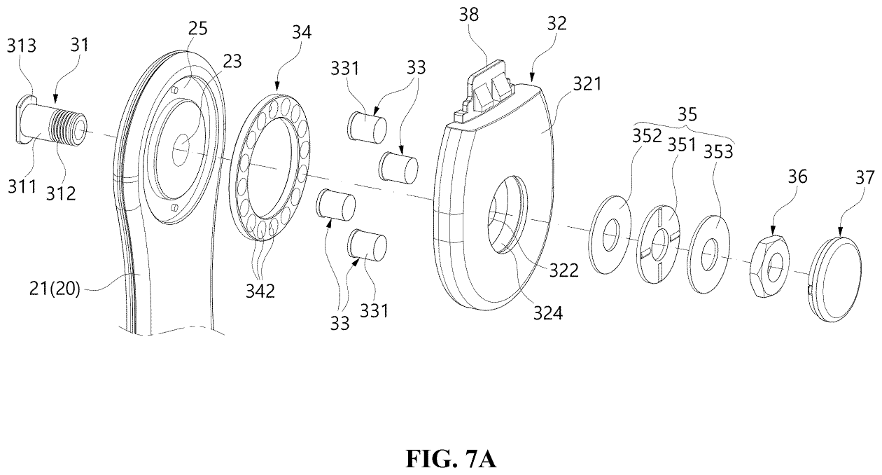

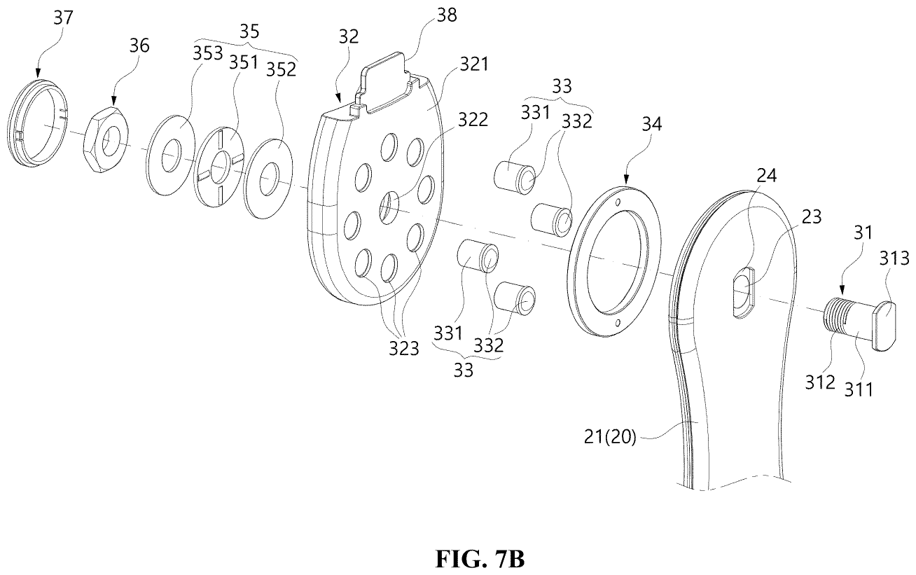

In addition, there may be provided a plurality of LED pads 10. In this case, the plurality of LED pads 10 may be different in the color of light irradiated from the LEDs arranged thereon. FIGS. 7A and 7B are enlarged exploded perspective views of a rotary unit in FIG. 2, in which FIG. 7A shows the rotary unit as seen from one side, and FIG. 7B shows the rotary unit as seen from the other side.

Referring to FIGS. 2, 7A and 7B, the rotary unit 30 includes a shaft 31 installed in the support unit 20, a spin block 32 rotatably installed on the shaft 31, a plurality of ball plungers 33 installed in the spin block 32 and spaced apart from each other along the inner circumference of the spin block 32, a ball spacer 34 installed in the support unit 20 to face the spin block 32 and having a plurality of engaging grooves 342 recessed on a ring-shaped body thereof to engage with balls 332 of the ball plungers 33, a thrust bearing 35 put on the shaft 31 and installed on the spin block 32 so as to allow the spin block 32 to spin, and a nut 36 fastened to the shaft 31 to prevent the thrust bearing 35 from coming off.

The shaft 31 is structured to have a threaded portion 312 formed on one side of a shaft body 311, and a retaining protrusion 313 formed at the other end.

The spin block 32 includes a shaft insertion hole 322 formed at the center of a block body 321, a plurality of plunger insertion portions 323 formed along the inner circumference of the shaft insertion hole 322 of the block body 321 so as to insert the ball plungers 33 therein, and a bearing mounting groove 324 recessed from the outer side of the block body 321 to receive the thrust bearing 35 therein. Meanwhile, the foregoing upper protrusion 38 protrudes from the upper portion of the block body 321.

The plurality of ball plungers 33 are equiangularly installed along the circumference of the shaft insertion hole 322 on the inner surface of the spin block 32 facing the ball spacer 34. Each ball plunger 33 includes a casing 331, and the ball 332 inserted and installed in the casing 331 so as to be retractable by an elastic member such as a spring (not shown) inserted in the casing 331.

Further, the ball plunger 33 is engaged with and released from the engaging groove 342 of the ball spacer 34 as the ball 332 moves forward/backward with the elastic force of the elastic member when the LED pad 10 rotates together with the rotary unit 30. In this case, the spin block 32 is locked at a specific position when the ball 322 is engaged with the engaging groove 342. Further, the spin block 32 becomes rotatable when the ball 322 is released from the engaging groove 342 by a rotational force applied to the spin block 32.

The thrust bearing 35 may be achieved by various well-known thrust bearings capable of rotatably supporting the spin block. However, the thrust bearing 35 according to this embodiment may include a roller assembly 351 in which a plurality of rollers are coupled to a disk, an inner ring 352 installed on the inner side of the roller assembly 351, and an outer ring 353 installed on the outer side of the roller assembly 351.

In addition, the rotary unit 30 is provided with a cover 37 installed to cover the nut 36. The cover 37 is provided in the form of a cap to cover and seal the nut 36, thereby improving safety and appearance.

Meanwhile, the support unit 20 includes a ring-shaped spacer groove 25 formed in an upper portion of a support bar 21 having a plate-like bar shape to receive the ball spacer 34, a shaft insertion hole 23 formed at the center of the spacer groove 25 to insert the shaft 31 therein, and a retaining groove 24 recessed in an outer portion of the shaft insertion hole 23 to receive the retaining protrusion 313 of the shaft 31.

Further, the support unit 20 includes the lower protrusion 22 in the lower portion thereof to couple with the base unit 40.

The base unit 40 includes the plurality of upper grooves 41 formed on the upper portion of a base body shaped like a bar so that the lower protrusion 22 of the support unit 20 can be position-variably fixed as press-fitted therein. The upper groove 41 has a rectangular, elliptical or similar shape to match the shape of the lower protrusion 22. The plurality of upper grooves 41 not only allows the position of the support unit 20 to be adjusted forward and backward, but also allows the plurality of LED pads 10 to be installed.

The base units 40 are provided as a pair to face each other and are connected by the fixing bar 50, thereby enabling the adjustment of a lateral distance between them. The pair of base units 40 are formed with lower grooves 42 in front and rear lower portions thereof so that the fixing bar 50 can be inserted therein. Here, the lower groove 42 is recessed in a T-shape.

Below, the operations of the position-variable LED light therapy device according to an embodiment of the disclosure will be described in brief.

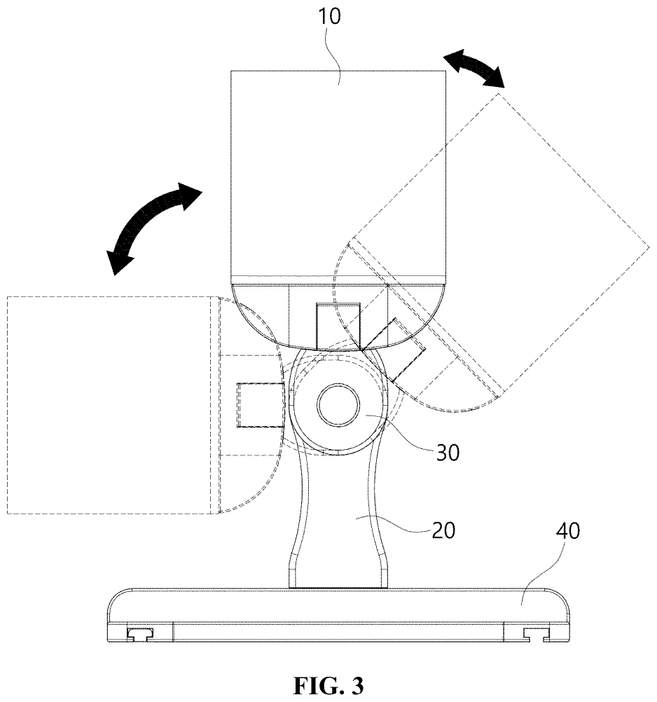

FIG. 3 is a view illustrating that a rotation angle of an LED pad is adjusted in a position-variable LED light therapy device according to an embodiment of the disclosure. As shown therein, the LED pad 10 is rotated at various angles, such as 22.5°, 45°, and 90° in accordance with the rotation of the rotary unit 30, and thus adjusted in angle to irradiate light onto a desired area.

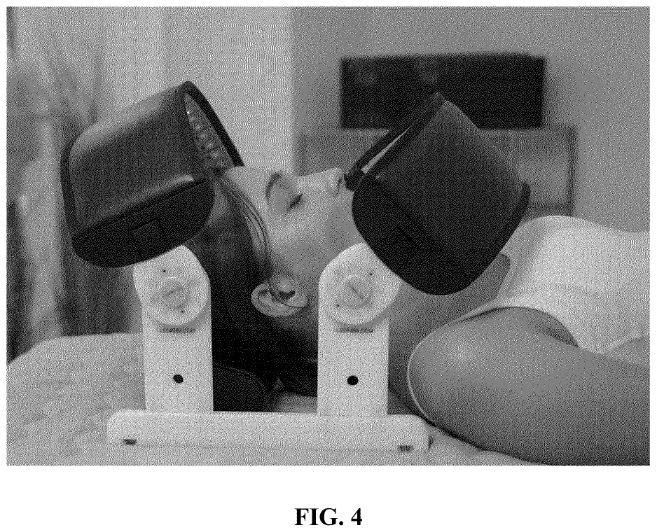

FIG. 4 is a view showing a state of using a position-variable LED light therapy device according to an embodiment of the disclosure. As shown therein, the pair of support units 20 are installed in front and rear positions of the base unit 40, and the LED pads 10 are respectively installed in the support units 20 with the rotary units 30 adjusted to irradiate light toward neck and head areas of the human body, so that the light can be irradiated to the two areas simultaneously, thereby shortening treatment time and enhancing therapeutic effects.

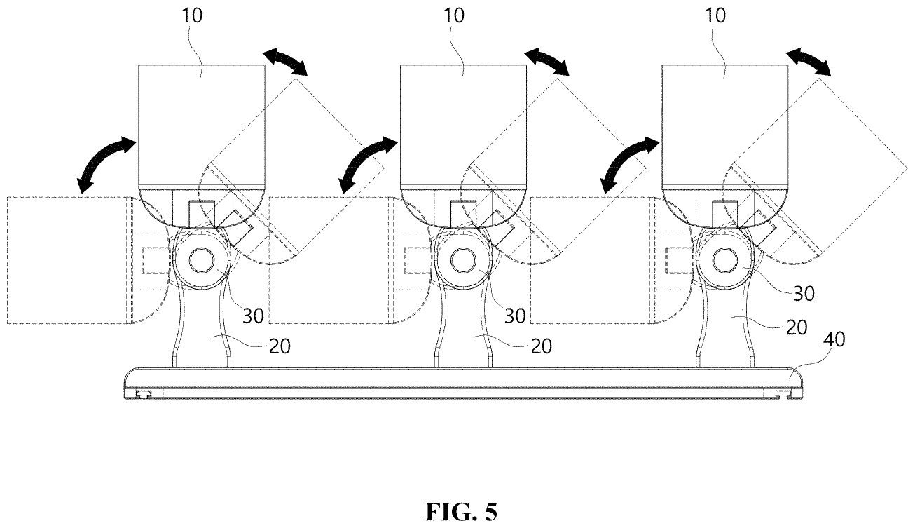

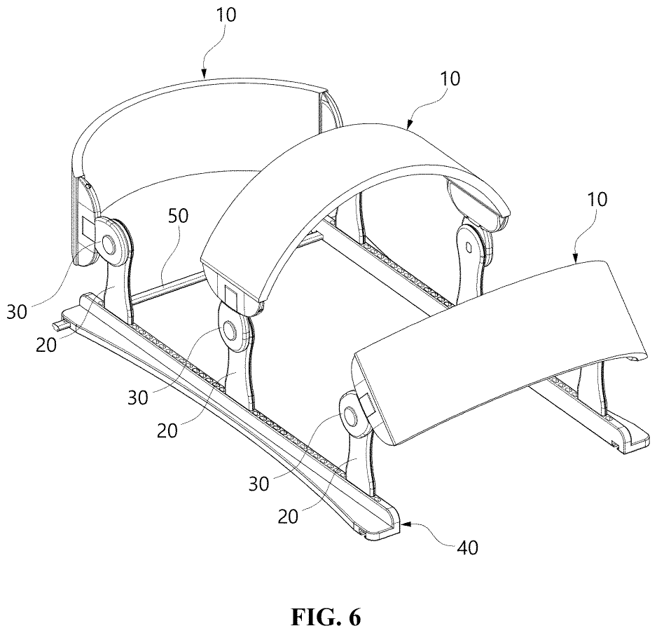

FIG. 5 is a side view showing another state of using a position-variable LED light therapy device according to an embodiment of the disclosure, and FIG. 6 is a perspective view showing another state of using a position-variable LED light therapy device according to an embodiment of the disclosure.

Referring to FIGS. 5 and 6, the position-variable LED light therapy device according to the disclosure may be used in such a manner that three pairs of support units 20 are installed on the base unit 40 and the LED pads 10 are respectively installed on the support units 20. By installing the three LED pads 10 in this way, light can be irradiated simultaneously at appropriate angles to more areas. In this case, when the plurality of LED pads 10 are different in the color of light irradiated from the LEDs arranged thereon, color therapy effects are expected by irradiating the lights of different colors and stimulating the human body by the unique wavelengths of these different colored lights.

Unless explicitly described to the contrary, the term “include,” “comprise” or “have” as used above mean the presence of the stated elements, and it is thus construed to further include other elements rather than precluding those elements. Unless otherwise defined, all terms including technical or scientific terms used herein have the same meaning as generally understood by a person having ordinary knowledge in the art to which the disclosure pertains. General terms, such as terms defined in the dictionary, should be construed as consistent in the contextual meaning of the relevant art, and are not be interpreted as having an idealistic or excessively formalistic meaning unless explicitly in the disclosure.

Although the configurations and operations of an unmanned cultivation robot system for controlled agriculture according to an embodiment of the disclosure have been described so far, it will be appreciated by those skilled in the art that modifications or substitutions may be made in all or part of the embodiments of the disclosure without departing from the technical spirit of the disclosure.

Accordingly, it will be understood that the scope of the disclosure falls into the appended claims and equivalents thereof.

### INDUSTRIAL APPLICABILITY

Disclosed is a position-variable LED light therapy device, in which a plurality of LED pads is adjustable in angle to enable convenient irradiation of multiple beams of light toward various areas and simultaneous irradiation of light toward multiple areas, thereby being applicable for therapeutic purposes such as treating damaged skin through selective regeneration or destruction of skin tissue, as well as for cosmetic purposes such as whitening, based on the principle that light irradiated onto the skin of the human body promotes biochemical reactions within the skin.

1. A position-variable light emitting diode (LED) light therapy device comprising an LED pad 10 on which a plurality of LEDs are arranged, a pair of support units 20 that supports the LED pad, a pair of base units 40 in which the pair of support units are installed, and a fixing bar 50 that secures the pair of base units spaced apart at a predetermined distance from each other, wherein
the support unit is provided with a rotary unit 30 in an upper portion thereof to adjust an installation angle of the LED pad, and
the base unit 40 is formed with a plurality of upper grooves 41 on an upper portion thereof to insert and fix the support unit 20 therein at various positions.

2. The position-variable LED light therapy device of claim 1, wherein
the rotary unit 30 includes an upper protrusion 38 formed to be inserted and fixed into a groove of the LED pad 10, and
the support unit 20 includes a lower protrusion 22 formed at a lower end thereof to be press-fitted into the upper groove.

3. The position-variable LED light therapy device of claim 1, wherein the LED pad 10 is configured to be separated from the rotary unit and to irradiate light manually to a desired area.

4. The position-variable LED light therapy device of claim 1, wherein the LED pad 10 comprises a plurality of LED pads 10, and the plurality of LED pads are different from each other in color of light irradiated from the LEDs arranged thereon.

5. The position-variable LED light therapy device of claim 1, wherein the plurality of support units are installed on the base unit 40 to install the plurality of LED pads.

6. The position-variable LED light therapy device of claim 1, wherein the rotary unit 30 comprises:
a shaft 31 installed in the support unit;
a spin block 32 rotatably installed on the shaft;
a plurality of ball plungers 33 installed in the spin block 32, and spaced apart from each other along the inner circumference of the spin block, each ball plunger comprising a casing, and a ball inserted and installed in the casing so as to be retractably by an elastic member;
a ball spacer 34 installed in the support unit to face the spin block 32 and comprising a plurality of engaging grooves recessed along the circumference thereof to engage with the balls of the ball plungers,
a thrust bearing 35 put on the shaft and installed on the spin block so as to allow the spin block 32 to spin, and
a nut 36 fastened to the shaft to prevent the thrust bearing from coming off.

7. The position-variable LED light therapy device of claim 6, wherein the rotary unit 30 comprises a cover 37 installed to cover the nut.

8. The position-variable LED light therapy device of claim 6, wherein
the shaft 31 is structured to comprise a threaded portion 312 formed on one side of a shaft body, and a retaining protrusion 313 formed at the other end,
the support unit 20 comprises a ring-shaped spacer groove 25 formed in an upper portion of a support bar 21 having a plate-like bar shape and recessed to receive the ball spacer 34, a shaft insertion hole 23 formed at the center of the spacer groove 25 to insert the shaft therein, and a retaining groove 24 recessed in an outer portion of the shaft insertion hole of the support unit to receive the retaining protrusion 313, and
the spin block 32 comprises a shaft insertion hole 322 formed at the center of a block body 321, a plurality of plunger insertion portions 323 formed along the inner circumference of the shaft insertion hole of the block body 321 so as to insert the ball plungers 33 therein, a bearing mounting groove 324 recessed from the outer side of the block body to insert the thrust bearing 35 therein, and the upper protrusion 38 protrudes from the upper portion of the block body.

9. The position-variable LED light therapy device of claim 1, wherein the base units 40 are provided as a pair to face each other, and the base unit is formed with a pair of lower grooves 42 in lower portions thereof to insert the fixing bar 50 therein.

10. The position-variable LED light therapy device of claim 9, wherein the lower groove 42 is recessed in a T-shape.
